(19) United States
(12) Patent Application Publication
(10) Pub. No.: US 2019/0300606 A1
(43) Pub. Date: Oct. 3, 2019

(54) Inhibitory Immune Receptor Inhibition Methods and Compositions

(71) Applicant: The Board of Trustees of the Leland Stanford Junior University, Stanford, CA (US)

(72) Inventors: Elliot C. Woods, Burlingame, CA (US); Han Xiao, East Palo Alto, CA (US); Carolyn R. Bertozzi, Stanford, CA (US)

(21) Appl. No.: 16/307,428

(22) PCT Filed: Jun. 30, 2017
(86) PCT No.: PCT/US2017/040483

§ 371 Date: Dec. 5, 2018

Related U.S. Application Data
(60) Provisional application No. 62/357,653, filed on Jul. 1, 2016.

(51) Int. Cl. C07K 16/28; A61P 35/00; C07K 16/32; G01N 33/574

(52) U.S. Cl. CPC C07K 16/2803; A61P 35/00; C07K 16/2851; C07K 16/32; A61K 2039/505; G01N 33/57492; C07K 2317/732; C07K 2317/76; C07K 16/2887

(57) ABSTRACT

Provided are methods relating to the inhibition of inhibitory immune receptors. Aspects of the present disclosure include methods that include administering to an individual receiving an antibody therapy an inhibitory immune receptor inhibitor. Also provided are compositions and kits that find use, e.g., in practicing the methods of the present disclosure.

### CROSS-REFERENCE TO RELATED APPLICATIONS

This application claims the benefit of U.S. Provisional Patent Application No. 62/357,653 filed Jul. 1, 2016, which application is incorporated herein by reference in its entirety.

### STATEMENT REGARDING FEDERALLY SPONSORED RESEARCH

This invention was made with Government support under contract GM059907 awarded by the National Institutes of Health. The Government has certain rights in the invention.

### INTRODUCTION

Therapies that enhance the immune response to cancer are proving revolutionary in the fight against intractable tumors. Immune cells integrate signals from activating and inhibitory receptors to determine their response to a challenging target—activating signals alert them to the presence of pathology while inhibitory signals tell the cell that it has confronted a healthy “self”. Successful tumors evolve mechanisms to thwart immune cell recognition, often by overexpressing ligands for inhibitory receptors. This discovery has led to new therapeutic strategies aimed at blocking inhibitory immune cell signaling, as embodied in clinically approved T cell checkpoint inhibitors targeting PD-1 and CTLA-4. Ongoing pre-clinical studies have focused on combining therapies targeting multiple immunologic pathways. For example, antibodies against PD-1/PD-L1 in combination with those targeting other T cell checkpoint inhibitors demonstrate improved anti-tumor activity in syngeneic tumor models. A complement to these interventions are therapies targeting innate immune cells, particularly natural killer (NK) cells, macrophages and dendritic cells.

### SUMMARY

Provided are methods relating to the inhibition of inhibitory immune receptors. Aspects of the present disclosure include methods that include administering to an individual receiving an antibody therapy an inhibitory immune receptor inhibitor. Also provided are compositions and kits that find use, e.g., in practicing the methods of the present disclosure.

### BRIEF DESCRIPTION OF THE FIGURES

FIG. 1 shows data demonstrating the potentiation of trastuzumab-dependent cytotoxicity in BT-20 cells by treatment with Siglec blocking antibodies.

FIG. 2 shows data demonstrating the potentiation of rituximab-dependent cytotoxicity in Ramos cells by treatment with Siglec blocking antibodies.

FIG. 3 shows flow cytometry data demonstrating that BT-20 cells are rich in Siglec-7 and Siglec-9 ligands.

FIG. 4 shows flow cytometry data demonstrating that Ramos cells are rich in Siglec-7 and Siglec-9 ligands.

FIG. 5 shows data indicating that Siglec-7 ligand abundance predicts an increase in ADCC by a trastuzumab-sialidase conjugate. HER2 expression level is indicated by number of ‘+’s. ADCC potentiation is most pronounced for HER2-low cell line, e.g., as seen in comparing MDA-MB-231 cells to SKBR3 cells.

### DETAILED DESCRIPTION

Provided are methods relating to the inhibition of inhibitory immune receptors. Aspects of the present disclosure include methods that include administering to an individual receiving an antibody therapy an inhibitory immune receptor inhibitor. Also provided are compositions and kits that find use, e.g., in practicing the methods of the present disclosure.

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

As summarized above, methods relating to inhibitory immune receptor inhibition are provided. In certain aspects, the methods include administering to an individual receiving an antibody therapy an inhibitory immune receptor inhibitor. Details of such methods will now be described.

As used herein, an “inhibitory immune receptor” is a receptor present on an immune cell that negatively regulates an immune response. According to certain embodiments, the inhibitory immune receptor inhibitor inhibits an inhibitory immune receptor present on an immune cell selected from a natural killer (NK) cell, a macrophage, a monocyte, a neutrophil, a dendritic cell, a T cell, a B cell, a mast cell, a basophil, and an eosinophil.

Examples of inhibitory immune receptors which may be inhibited according to the methods of the present disclosure include inhibitory immune receptors of the Ig superfamily, including but not limited to: CD200R, CD300a (IRp60; mouse MAIR-I), CD300f (IREM-1), CEACAM1 (CD66a), FcγRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of inhibitory immune receptors which may be inhibited according to the methods of the present disclosure include sialic acid-binding Ig-like lectin (Siglec) receptors, including but not limited to: Siglec-2, Siglec-3 (CD33), Siglec-5, mouse Siglec-f, Siglec-6, Siglec-7, Siglec-8, Siglec-9, mouse Siglec-e, Siglec-10, mouse Siglec-g, Siglec-11, and Siglec-12. Additional examples of inhibitory immune receptors which may be inhibited according to the methods of the present disclosure include C-type lectins, including but not limited to: CLEC4A (DCIR), Ly49Q and MICL. Details regarding inhibitory immune receptors may be found, e.g., in Steevels et al. (2011) Eur. J. Immunol. 41(3):575-587.

In some embodiments, the inhibitory immune receptor is a receptor for which the ligand is selected from an oligosaccharide, a polysaccharide (or “glycan”, that is, a molecule containing monosaccharides linked glycosidically), a glycoprotein, a glycolipid, and a ganglioside. In certain aspects, the inhibitory immune receptor is a receptor for which the ligand has a terminal sialic acid residue. According to certain embodiments, the inhibitory immune receptor is a receptor for which the ligand is a sialoglycan.

According to certain embodiments, the methods of the present disclosure include administering two or more inhibitory immune receptor inhibitors to the individual receiving an antibody therapy. As just one example, the methods may include administering to the individual a Siglec-7 inhibitor and a Siglec-9 inhibitor (e.g., concurrently (e.g., as part of the same or different compositions) or sequentially).

By inhibitory immune receptor “inhibitor” is meant an agent that reduces or abolishes the biological activity of an inhibitory immune receptor. The inhibitor employed may vary depending upon the nature of the inhibitory immune receptor. Non-limiting examples of inhibitors that may be employed include small molecules, ligands, and antibodies.

According to certain embodiments, the inhibitor is a small molecule. As used herein, a “small molecule” is a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer.

In certain aspects, the inhibitor is an antibody. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the inhibitory immune receptor, including, but not limited to single chain Fv (scFv), Fab, F(ab′)₂, (scFv′)₂, and diabodies; chimeric antibodies; monoclonal antibodies, human antibodies, humanized antibodies (e.g., humanized whole antibodies, humanized half antibodies, or humanized antibody fragments); and fusion proteins comprising an antigen-binding portion of an antibody and a non-antibody protein. The antibodies may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

In certain aspects, the inhibitor (e.g., a small molecule, an antibody, a sialic acid derivative, etc.) inhibits the biological activity of an inhibitory immune receptor by binding (e.g., specifically binding) to the inhibitory immune receptor. As used herein, an inhibitor that “specifically binds” the inhibitory immune receptor or is “specific” for the inhibitory immune receptor refers to an inhibitor that binds the inhibitory immune receptor with greater affinity than with other receptors. According to certain embodiments, the inhibitor exhibits a binding affinity to the inhibitory immune receptor of a K_{d} of less than or equal to about 10⁻⁵ M, less than or equal to about 10⁻⁶ M, or less than or equal to about 10⁻⁷ M, or less than or equal to about 10⁻⁸ M, or less than or equal to about 10⁻⁹ M, 10⁻¹⁰M, 10⁻¹¹ M, or 10⁻¹² M or less. Such affinities may be readily determined using conventional techniques, such as by equilibrium dialysis, surface plasmon resonance (SPR) technology (e.g., using the BIAcore 2000 instrument, using general procedures outlined by the manufacturer), radioimmunoassay, or by another method.

In some embodiments, the inhibitor may be a known inhibitor of the inhibitory immune receptor of interest. In other embodiments, the inhibitor is identified, e.g., using a suitable approach for screening small molecules (e.g., by screening a combinatorial library of small molecules), antibodies (e.g., by phage or yeast display of antibody libraries), ligands, or the like for the ability to inhibit (e.g., by binding) the inhibitory immune receptor. The readout for such screening approaches will vary depending upon the inhibitory immune receptor of interest.

In certain aspects, the methods include administering a sialic acid-binding Ig-like lectin (Siglec) inhibitor to the individual receiving an antibody therapy. The Siglec inhibitor may be an inhibitor of, e.g., any of Siglecs 1-17. According to certain embodiments, the inhibitor inhibits Siglec-7 (UniProtKB-Q9Y286), Siglec-9 (UniProtKB-Q9Y336), or both. Such an inhibitor may be an antibody, a small molecule, a sialic acid derivative, or the like. Siglec inhibitors are described, e.g., in Cagnoni et al. (2016) Front. Oncol. 6:109, and include, e.g., Oxamido-Neu5Ac, BPC-Neu5Ac, BPC-Neu5Ac-Dox liposome, 9-BPC-4-mNPC-Neu5Ac, and the like. Rational structure-based Siglec-7 inhibitor design is described, e.g., in Attrill et al. (2006) Biochem. J. 397(2):271-8. High-affinity Siglec-7 inhibitors are described, e.g., in Prescher et al. (2017) J. Med. Chem. 60(3):941-956.

In some embodiments, the inhibitory immune receptor inhibitor is an antibody that inhibits Siglec-7, Siglec-9, or both. Non-limiting examples of available Siglec-7 and Siglec-9 blocking antibodies are provided in the Experimental section below. In certain aspects, the administered antibody that inhibits Siglec-7, Siglec-9, or both, is a polyclonal, monoclonal, humanized, fully human, asymmetric, or heteromeric antibody, or an antibody having any combination of such features to the extent possible. In some embodiments, the antibody that inhibits Siglec-7, Siglec-9, or both, is a whole antibody (e.g., an antibody composed of a tetramer which in turn is composed of two dimers of a heavy and light chain polypeptide), such as a whole IgG (e.g., IgG1, IgG2, IgG3 or IgG4), IgE, IgD, IgA, IgM, etc. antibody. In other aspects, the antibody that inhibits Siglec-7, Siglec-9, or both, is an antibody fragment, non-limiting examples of which are single chain Fv (scFv), Fab, F(ab′)₂, (scFv′)₂, and the like. The antibody that inhibits Siglec-7, Siglec-9, or both, may be a known antibody. In certain aspects, such an antibody is identified, e.g., using a suitable approach for screening antibodies (e.g., by phage or yeast display of antibody libraries), for the ability to bind Siglec-7, Siglec-9, or both.

Antibodies that specifically bind an inhibitory immune receptor of interest (e.g., Siglec-7, Siglec-9, or both) can be prepared using a wide variety of techniques known in the art including the use of hybridoma, recombinant, phage display technologies, or a combination thereof. For example, an antibody may be made and identified/produced using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds Siglec-7, Siglec-9, or both, can be selected or identified with Siglec-7 and/or Siglec-9, e.g., using labeled Siglec-7 and/or Siglec-9 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

By “antibody therapy” is meant that an antibody (which is not an inhibitory immune receptor inhibitor) will be, has been, and/or is being administered to the individual for a therapeutic purpose. The antibody therapy will vary depending upon the condition of the individual being treated. In some embodiments, the antibody therapy includes the administration of an antibody (e.g., an IgG1, IgG2, IgG3, or IgG4 antibody) that specifically binds to an antigen (e.g., a cell surface antigen, such as a protein or non-protein cell surface antigen) on the surface of a cell relevant to the medical condition of the individual. For example, the antibody administered as part of the antibody therapy may bind to an antigen present on the surface of a cell that contributes to the medical condition, where binding of the antibody to the antigen reduces or abolishes the cell's contribution to the medical condition. According to certain embodiments, the antibody therapy includes administering to the individual an antibody selected from trastuzumab, cetuximab, daratumumab, girentuximab, panitumumab, ofatumumab, and rituximab.

In certain aspects, the individual is receiving an antibody therapy that includes administering to the individual an antibody that induces antibody-dependent cellular cytotoxicity (ADCC). ADCC is the killing of an antibody-coated target cell by a cytotoxic effector cell (e.g., via a nonphagocytic process), characterized by the release of the content of cytotoxic granules and/or by the expression of cell death-inducing molecules. ADCC may be triggered through interaction of target-bound antibodies (e.g., IgG (e.g., IgG1, IgG2, IgG3, or IgG4), IgA, or IgE antibodies) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). ADCC involving human IgG1, the most used IgG subclass for therapeutic antibodies, has been shown to be dependent on the glycosylation profile of its Fc portion and on the polymorphism of Fcγ receptors.

Non-limiting examples of antibodies that may be administered to the individual as part of the antibody therapy include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof. As used herein, “variant” is meant the antibody binds to the target/antigen (e.g., HER2 for trastuzumab) but has fewer or more amino acids than the parental antibody, has one or more amino acid substitutions relative to the parental antibody, or a combination thereof.

In some embodiments, the individual is receiving an antibody therapy that includes administering to the individual an antibody set forth in Table 1 below approved for treating cancer, or an antigen-binding variant thereof. Also provided in Table 1 is the corresponding tumor-associated antigen or tumor-specific antigen to which the therapeutic antibody specifically binds, as well as the type of cancer for which the antibody is approved for treatment.

TABLE 1
Antibodies approved for treating cancer

| Antigen | Cancer Types | Antibody |
|---|---|---|
| BCR-ABL | CML | Imatinib, Dasatinib |
| | ALL | Nilotinib, Bosutinib |
| | | Ponatinib |
| CD19 | ALL | Blinatumomab |
| CD20 | NHL, CLL | Rituximab |
| | B-cell NHL | Ofatumumab |
| | pre-B ALL | ⁹⁰Y-Ibritumomab |
| | | ¹³¹I-Tositumomab |
| CD30 | Hodgkin's lymphoma | Brentuximab vedotin |
| CD33 | AML | Gemtuzumab |
| | | ozogamicin |
| CD52 | CLL | Alemtuzumab |
| CTLA-4 | Unresectable or metastatic | Ipilimumab |
| | melanoma | |
| EGFR | CRC | Cetuximab |
| | Head and Neck | Panitumumab |
| EpCAM | Malignant ascites | Catumaxomab |
| HER2 | Breast | Trastuzumab |
| | | Pertuzumab |
| PAP | Prostate | Sipuleucel-T |
| PD-1 | Metastatic melanoma NSCLC | Nivolumab |
| | | Pembrolizumab |
| VEGF | Breast, Cervical | Bevacizumab |
| | CRC, NSCLC | |
| | RCC, Ovarian | |
| | Glioblastoma | |
| VEGF-R2 | Gastric | Ramucirumab |
| | NSCLC | |

Abbreviations for Table 1 are as follows: ALL, acute lymphoblastic leukemia; AML, acute myelogenous leukemia; BCR-ABL, breakpoint cluster region Abelson tyrosine kinase; CLL, chronic lymphocytic leukemia; CTLA-4, cytotoxic T-lymphocyte-associated antigen 4; CRC, colorectal cancer; EGFR, epidermal growth factor receptor; EpCAM, epithelial cell adhesion molecule; HER2, human epidermal growth factor receptor 2; NHL, non-Hodgkin's lymphoma; NSCLC, non-small cell lung cancer; PAP, prostatic acid phosphatase; PD-1, programmed cell death receptor 1; RCC, renal cell carcinoma; VEGF, vascular endothelial growth factor; VEGF-R2, vascular endothelial growth factor receptor 2.

In certain aspects, the individual is receiving an antibody therapy that includes administering to the individual an antibody set forth in Table 2 below or an antigen-binding variant thereof. Also provided in Table 2 is the corresponding tumor-associated antigen or tumor-specific antigen to which the therapeutic antibody specifically binds, as well as an example cancer type which may be treated using the antibody.

TABLE 2
Additional antibodies, cell surface molecules, and cancer types

| Antigen | Cancer Types | Antibody |
|---|---|---|
| A2aR | NSCLC | PBF-509 |
| AKAP4 | NSCLC | Preclinical |
| | Ovarian | |
| BAGE | Glioblastoma | Preclinical |
| | Ovarian | |
| BORIS | Prostate, Lung | Preclinical |
| | Esophageal | |
| CD22 | ALL | Epratuzumab |
| | | Moxetumomab |
| | | Inotuzumab ozogamicin |
| CD73 | Advanced solid tumors | MEDI9447 |
| CD137 | Advanced solid tumors | Urelumab |
| | | PF-05082566 |
| CEA | CRC | PANVAC ™ |
| | | Ad5-[E1-, E2b-]-CEA(6D) |
| CS1 | Multiple myeloma | Elotuzumab |
| CTLA-4 | Malignant mesothelioma | Tremelimumab |
| EBAG9 | Bladder | Preclinical |
| EGF | NSCLC | CIMAvax |
| EGFR | NSCLC | Necitumumab |
| GAGE | Cervical | Preclinical |
| GD2 | Neuroblastoma | Dinutuximab, hu3F8 |
| | Retinoblastoma | hu14.18-IL-2, |
| | | 3F8/OKT3BsAb |
| | Melanoma other | anti-GD2 CAR |
| | solid tumors | GD2-KLH |
| gp100 | Melanoma | gp100: 209-217(210M) |
| HPV-16 | Cervical | HPV-16 (E6, E7) |
| | SCCHN | TG4001, Lm-LLO-E7 |
| | | pNGVL4a-CRT/E7, |
| | | INO-3112 |
| HSP105 | CRC | Preclinical |
| | Bladder | |
| IDH1 | Glioma | IDH1(R132H) p123-142 |
| Idiotype | NSCLC, Breast | Racotumomab |
| (NeuGcGM3) | Melanoma | |
| IDO1 | Breast, Melanoma | Indoximod |
| | NSCLC | INCB024360 |
| | | IDO1 peptide vaccine |
| KIR | Lymphoma | Lirilumab |
| LAG-3 | Breast, Hematological, | BMS-986016 |
| | Advanced solid tumors | IMP321 |
| LY6K | Gastric | LY6K-177 peptide |
| | SCCHN | LY6K, CDCA1, IMP3 |
| MAGE-A3 | Melanoma | recMAGE-A3 |
| | NSCLC | Zastumotide |
| MAGE-C2 | Gastric, Melanoma | Preclinical |
| | Multiple myeloma | |
| MAGE-D4 | CRC | Preclinical |
| Melan-A | Melanoma | MART-1 (26-35, 27L) |
| MET | NSCLC | Onartuzumab |
| | | Tivantinib |
| MUC1 | NSCLC, Breast | Tecemotide, TG4010 |
| | Prostate | PANVAC ™ |
| MUC4 | Pancreatic | Preclinical |
| MUC16 | Ovarian | Abagovomab |
| | | Oregovomab |
| NY-ESO-1 | Ovarian | NY-ESO-1/ISCOMATRIX ™ |
| | Melanoma | rV-NY-ESO-1; rF-NY-ESO-1 |
| PD-1 | B-cell lymphoma | Pidilizumab |
| | Melanoma, CRC | AMP-224, AMP-514 |
| PD-L1 | NSCLC, RCC | BMS-936559, Atezolizumab |
| | Bladder, Breast | Durvalumab, Avelumab |
| | Melanoma, SCCHN | |
| PRAME | NSCLC | Preclinical |
| PSA | Prostate | PROSTVAC ®-VF |
| ROR1 | CLL, Pancreatic | Preclinical |
| | Lung, Breast | |
| Sialyl-Tn | Breast | Theratope |
| SPAG-9 | Prostate, CRC | Preclinical |
| | NSCLC, Ovarian | |
| SSX1 | Prostate | Preclinical |
| | Multiple myeloma | |
| Survivin | Melanoma | EMD640744 |
| | Glioma, Solid tumors | Trivalent peptide vaccine |
| | | Tripeptide vaccine |
| Telomerase | Pancreatic | Tertomotide |
| TIM-3 | Melanoma, NHL NSCLC | Preclinical |
| VISTA | Melanoma, Bladder | Preclinical |
| WT1 | Ovarian, Uterine, AML | WT1 peptide vaccine |
| | Multiple myeloma | |
| XAGE-1b | Prostate | DC-based tumor vaccine |
| 5T4 | RCC, CRC | TroVax ® |
| | Prostate | Naptumomab estafenatox |

Abbreviations for Table 2 are as follows: A2aR, adenosine A2a receptor; AKAP4, A kinase anchor protein 4; AML, acute myelogenous leukemia; ALL, acute lymphoblastic leukemia; BAGE, B melanoma antigen; BORIS, brother of the regulator of imprinted sites; CEA, carcinoembryonic antigen; CLL, chronic lymphocytic leukemia; CRC, colorectal cancer; CS1, CD2 subset 1; CTLA-4, cytotoxic T-lymphocyte-associated antigen 4; EBAG9, estrogen receptor binding site associated antigen 9; EGF, epidermal growth factor; EGFR, epidermal growth factor receptor; NSCLC, non-small cell lung cancer; GAGE, G antigen; GD2, disialoganglioside GD2; gp100, glycoprotein 100; HPV-16, human papillomavirus 16; HSP105, heat-shock protein 105; IDH1, isocitrate dehydrogenase type 1; IDO1, indoleamine-2,3-dioxygenase 1; KIR, killer cell immunoglobulin-like receptor; LAG-3, lymphocyte activation gene 3; LY6K, lymphocyte antigen 6 complex K; MAGE-A3, melanoma antigen 3; MAGE-C2, melanoma antigen C2; MAGE-D4, melanoma antigen D4; Melan-A/MART-1, melanoma antigen recognized by T-cells 1; MET, N-methyl-N′-nitroso-guanidine human osteosarcoma transforming gene; MUC1, mucin 1; MUC4, mucin 4; MUC16, mucin 16; NHL, non-Hodgkin lymphoma; NY-ESO-1, New York esophageal squamous cell carcinoma 1; PD-1, programmed cell death receptor 1; PD-L1, programmed cell death receptor ligand 1; PRAME, preferentially expressed antigen of melanoma; PSA, prostate specific antigen; RCC, renal cell carcinoma; ROR1, receptor tyrosine kinase orphan receptor 1; SCCHN, squamous cell carcinoma of the head and neck; SPAG-9, sperm-associated antigen 9; SSX1, synovial sarcoma X-chromosome breakpoint 1; TIM-3, T-cell immunoglobulin domain and mucin domain-3; VISTA, V-domain immunoglobulin-containing suppressor of T-cell activation; WT1, Wilms' Tumor-1; XAGE-1 b, X chromosome antigen 1b.

In some embodiments, the individual is receiving an antibody therapy that includes administering to the individual an antibody set forth in Table 3 below, or a variant thereof. Also provided in Table 3 is the corresponding tumor-associated antigen or tumor-specific antigen to which the therapeutic antibody specifically binds.

TABLE 3
Additional antibodies and corresponding cell surface molecules

| Antibody | Antigen |
|---|---|
| oregovomab | CA125 |
| girentuximab | CAIX |
| obinutuzumab | CD20 |
| ofatumumab | CD20 |
| rituximab | CD20 |
| alemtuzumab | CD52 |
| ipilimumab | CTLA-4 |
| tremelimumab | CTLA-4 |
| cetuximab | EGFR |
| necitumumab | EGFR |
| panitumumab | EGFR |
| zalutumumab | EGFR |
| edrecolomab | EpCAM (17-1A) |
| farletuzumab | FR-alpha |
| pertuzumab | Her2 |
| trastuzumab | Her2 |
| rilotumumab | HGF |
| figitumumab | IGF-1 |
| ganitumab | IGF1R |
| durvalumab | IGG1K |
| bavituximab | Phosphatidylserine |
| onartuzumab | scatter factor receptor kinase |
| bevacizumab | VEGF-A |
| ramucirumab | VEGFR2 |

In certain aspects, the individual is receiving an antibody therapy that includes administering to the individual an antibody selected from trastuzumab, cetuximab, daratumumab, girentuximab, panitumumab, ofatumumab, rituximab, and variants thereof.

According to certain embodiments, the antibody administered as part of the antibody therapy may be conjugated to an agent, e.g., a therapeutic agent, a labeling agent (e.g., an in vivo imaging agent), or the like. For example, the antibody may be part of an antibody-drug conjugate (ADC). Drugs of interest include agents capable of affecting the function of a cell/tissue to which the conjugate binds via specific binding of the antibody portion of the conjugate to an antigen on the surface of the cell/tissue. For example, the agent may boost the function of the cell/tissue to which the conjugate specifically binds. Alternatively, when the function of the cell/tissue is pathological, an agent that reduces the function of the cell/tissue may be employed. In certain aspects, a conjugate includes an agent that reduces the function of a target cell/tissue by inhibiting cell proliferation and/or killing the cell/tissue. Such agents may vary and include cytostatic agents and cytotoxic agents, e.g., an agent capable of killing a target cell tissue with or without being internalized into a target cell.

In certain aspects, the antibody administered as part of the antibody therapy is conjugated to a drug selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

The type of individual receiving the antibody therapy may vary. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

According to certain embodiments, the individual has cancer. In certain aspects, the individual has a cancer set forth in Table 1 or Table 2 above. In some embodiments, the cancer is selected from breast cancer, ovarian cancer, gastric cancer, colon cancer, and renal carcinoma. When the individual has cancer, the antibody therapy may include administering to the individual an antibody that binds to an antigen present on a cancer cell of the individual. In some embodiments, the antibody binds to an antigen set forth in Table 1, Table 2, or Table 3 above. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In certain aspects, the cancer cell is a carcinoma cell. According to certain embodiments, the cancer cell is selected from a breast cancer cell, an ovarian cancer cell, a gastric cancer cell, a colon cancer cell, and a renal carcinoma cell.

In certain aspects, when the individual has cancer, the antibody therapy includes administering to the individual an antibody (e.g., an ADCC-inducing antibody) that binds to a tumor-associated antigen or a tumor-specific antigen. By “tumor-associated antigen” is meant an antigen expressed on malignant cells with limited expression on cells of normal tissues, an antigen expressed at much higher density on malignant versus normal cells, or an antigen that is developmentally expressed. In certain aspects, the antibody therapy includes administering to the individual an antibody that binds to a tumor-associated antigen or a tumor-specific antigen selected from HER2, CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), neuropilin-1 (NRP1), matriptase, or any other tumor-associated or tumor-specific antigens of interest. In some embodiments, the antibody therapy includes administering to the individual an antibody set forth in any of Table 1, Table 2, or Table 3 above, or an antigen-binding variant thereof.

According to certain embodiments, the methods of the present disclosure include determining the abundance of one or more inhibitory immune receptor ligands present on cells targeted by the antibody therapy prior to administering the inhibitory immune receptor inhibitor. Ligand abundance may be determined using any suitable approach and may vary depending upon the type of ligand to be detected.

In certain aspects, ligand abundance is determined in vivo (that is, in the individual). Approaches for detecting molecules (e.g., proteins) of interest in vivo are known and include, e.g., in vivo imaging. For example, when the identity of the ligand is known, an agent (e.g., an antibody) labeled with (e.g., conjugated to) an in vivo imaging agent may be administered to the individual, followed by detection of the ligand (via a detectable label of the imaging agent) to determine the location and abundance of the ligand. Suitable in vivo imaging agents include, but are not limited to, those that find use in in vivo imaging applications such as near-infrared (NIR) imaging, single photon emission computed tomography (SPECT), and/or the like. Details regarding suitable in vivo imaging approaches may be found, e.g., in Tunnell, J. In Vivo Clinical Imaging and Diagnosis ISBN: 9780071626835.

In some embodiments, the abundance of one or more inhibitory immune receptor ligands present on cells targeted by the antibody therapy is determined in vitro. For example, ligand abundance may be determined on a biopsy sample, e.g., on target cells/tissue removed from the individual. Any suitable in vitro approach to determine the abundance of the one or more inhibitory immune receptor ligands may be employed. Such approaches include, but are not limited to, flow cytometry, enzyme-linked immunosorbent assays (ELISA), immunofluorescence, immunohistochemistry, etc. In certain aspects, an antibody that specifically binds the ligand is employed. In other aspects, a detection reagent that includes all or a portion of an extracellular domain of the inhibitory immune receptor is employed. For example, all or a portion of an extracellular domain of the inhibitory immune receptor may be part of a soluble fusion protein. Such a fusion protein may include all or a portion of an extracellular domain of the inhibitory immune receptor fused to a protein (e.g., a fragment crystallizable (Fc) antibody fragment, a protein tag, etc.) to which a secondary detection reagent may bind. The secondary detection reagent may be a labeled secondary antibody, such as an antibody labeled with a fluorescent dye, a radioisotope, an enzyme which generates a detectable product, or the like.

In certain aspects, the inhibitory immune receptor inhibitor is administered to the individual only upon determining that the abundance of the one or more inhibitory immune receptor ligands on cells targeted by the antibody therapy exceeds a threshold abundance level. The threshold abundance level may be based on one or more criteria. For example, when the ligand is detected using a labeled detection reagent (e.g., a fluorescently-labeled reagent), the threshold abundance level may be based at least in part on the signal (e.g., fluorescence) intensity as compared to a control. Fluorescence intensity, for example, may be determined by flow cytometry, immunofluorescence staining, or the like. Suitable controls to which the signal intensity may be compared include, e.g., the signal intensity from a different labeled reagent (e.g., a different fluorophore) relating to a different molecule, e.g., which is known to not significantly vary in abundance between normal cells and the type(s) of cells being evaluated in the biopsy sample. In another example, the signal intensity relating to the ligand on the surface of abnormal cells from the individual may be compared to the fluorescence intensity relating to the same ligand on the surface of control cells, which control cells may be, e.g., counterpart normal cells from the individual, counterpart normal cells from a different individual, cells from a cell line for which the abundance of the ligand is stable and has been established, or the like.

The threshold abundance level may be a ratio of the signal intensity relating to the inhibitory immune receptor ligand from cells in the biopsy sample to a signal intensity relating to a control molecule from cells in the biopsy sample. In certain aspects, the threshold abundance level may be a ratio (e.g., 1:1, 1.5:1, 2:1, 2.5:1, 3:1, etc.) of the signal intensity relating to the inhibitory immune receptor ligand from cells in the biopsy sample to a signal intensity relating to the inhibitory immune receptor ligand from control cells.

According to certain embodiments, the inhibitory immune receptor inhibitor is administered to the individual only upon determining that the abundance of the antigen to which the antibody of the therapy binds exceeds a threshold level. In some embodiments, the inhibitory immune receptor inhibitor is administered to the individual only upon determining that the abundance of the antigen to which the antibody of the therapy binds is less than a threshold level. For example, the present inventors have found that—in some instances (e.g., for certain therapeutic antibodies)—the benefits of administering the inhibitory immune receptor inhibitor are greatest when the abundance level of the antigen to which the antibody of the therapy binds is moderate or low. One such example is provided in the Experimental section below.

As summarized above, the inhibitory immune receptor inhibitor is administered to an individual receiving an antibody therapy. According to certain embodiments, the inhibitory immune receptor inhibitor is administered to the individual prior to the onset of the antibody therapy, concurrently with the antibody therapy, or both.

According to certain embodiments, the antibody administered to the individual as part of the antibody therapy and/or the inhibitory immune receptor inhibitor are administered according to a dosing regimen approved for individual use. In some embodiments, the administration of the inhibitory immune receptor inhibitor permits the antibody administered to the individual as part of the antibody therapy to be administered according to a dosing regimen that involves one or more lower and/or less frequent doses, and/or a reduced number of cycles as compared with that utilized when the antibody is administered without administration of the inhibitory immune receptor inhibitor.

In certain aspects, one or more doses of the antibody administered to the individual as part of the antibody therapy and the inhibitory immune receptor inhibitor are administered at the same time; in some such embodiments, such agents may be administered present in the same pharmaceutical composition. In some embodiments, however, the antibody administered to the individual as part of the antibody therapy and the inhibitory immune receptor inhibitor are administered to the individual in different compositions and/or at different times. For example, the antibody administered to the individual as part of the antibody therapy may be administered prior to administration of the inhibitory immune receptor inhibitor (e.g., in a particular cycle). Alternatively, the inhibitory immune receptor inhibitor may be administered prior to administration of the antibody of the antibody therapy (e.g., in a particular cycle). The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the inhibitory immune receptor inhibitor is administered to the individual for a desirable period of time prior to administration of the antibody that is part of the antibody therapy. In certain aspects, such a regimen “pre-blocks” the biology activity of the inhibitory immune receptor to potentiate ADCC resulting from the subsequent administration of an antibody that induces ADCC. Such a period of time separating a step of administering the inhibitory immune receptor inhibitor from a step of administering the antibody of the antibody therapy is of sufficient length to permit inhibition of the target inhibitory immune receptor, desirably so that ADCC mediated by the antibody of the antibody therapy is increased.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

By way of example, the antibody of the antibody therapy may be administered a period of time after administration of the inhibitory immune receptor inhibitor. The period of time may be selected to be correlated with inhibition of the inhibitory immune receptor by the inhibitory immune receptor inhibitor. In certain aspects, the relevant period of time permits (e.g., is correlated with) inhibition of the inhibitory immune receptor to a level that is 90% or less, 80% or less, 70% or less, 60% or less, 50% or less, 40% or less, 30% or less, 20% or less, or 10% or less than that observed on the relevant immune cells (e.g., NK cells) prior to (or at the moment of) the administration of the antibody of the antibody therapy.

In some embodiments, the inhibitory immune receptor inhibitor and the antibody of the antibody therapy are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating cellular cytotoxicity dependent upon the antibody of the antibody therapy), may include: (a) a first dosing period during which a therapeutically effective amount a first agent is administered to a patient; (b) a first resting period; (c) a second dosing period during which a therapeutically effective amount of a second agent and, optionally, a third agent, is administered to the patient; and (d) a second resting period. By “therapeutically effective amount” is meant a dosage sufficient to produce a desired result, e.g., an amount sufficient to effect beneficial or desired therapeutic (including preventative) results, such as a reduction in a symptom of a disease or disorder associated with the target cell or a population thereof, as compared to a control. An effective amount can be administered in one or more administrations.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event (e.g., inhibition of the inhibitory immune receptor), then the second resting period's length may be determined on the basis of different factors, separately or in combination. Exemplary such factors may include type and/or stage of a cancer against which an anti-tumor antibody therapy is administered; identity and/or nature of a targeted tumor antigen, identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The antibody that is administered as part of the antibody therapy and the inhibitory immune receptor inhibitor may be administered together or independently via any suitable route of administration. Such agents may be administered via a route of administration independently selected from oral, parenteral (e.g., by intravenous, intra-arterial, subcutaneous, intramuscular, or epidural injection), topical, or nasal administration. According to certain embodiments, antibody that is administered as part of the antibody therapy and the inhibitory immune receptor inhibitor are both administered parenterally, either concurrently (in the same pharmaceutical composition or separate pharmaceutical compositions) or sequentially.

In certain aspects, the methods include administering to the individual a further therapeutic agent in addition to the antibody that is administered as part of the antibody therapy and the inhibitory immune receptor inhibitor. Such administration may include concurrently administering the further therapeutic agent and one or both of the antibody that is administered as part of the antibody therapy and the inhibitory immune receptor inhibitor, or administering the further therapeutic agent sequentially with respect to one or both of the antibody that is administered as part of the antibody therapy and the inhibitory immune receptor inhibitor. In some embodiments, the individual has cancer, and the further therapeutic agent is an anti-cancer agent. Anti-cancer agents of interest include, but are not limited to, anti-cancer antibodies (e.g., any of the antibodies set forth in Tables 1, 2, and 3 above), small molecule anti-cancer agents, or the like.

In some embodiments, the further therapeutic agent is a small molecule anti-cancer agent selected from abiraterone, bendamustine, bexarotene, bortezomib, clofarabine, decitabine, exemestane, temozolomide, afatinib, axitinib, bosutinib, cabozantinib, crizotinib, dabrafenib, dasatinib, erlotinib, gefitinib, ibrutinib, imatinib, lapatinib, nilotinib, pazopanib, ponatinib, regorafenib, ruxolitinib, sorafenib, sunitinib, vandetanib, vemurafenib, enzalutamide, fulvestrant, epirubicin, ixabepilone, nelarabine, vismodegib, cabazitaxel, pemetrexed, azacitidine, carfilzomib, everolimus, temsirolimus, eribulin, omacetaxine, trametinib, lenalidomide, pomalidomide, romidepsin, vorinostat, brigatinib, ribociclib, midostaurin, telotristat ethyl, niraparib, cabozantinib, lenvatinib, rucaparib, granisetron, dronabinol, venetoclax, alectinib, cobimetinib, panobinostat, palbociclib, talimogene laherparepvec, lenvatinib, trifluridine and tipiracil, ixazomib, sonidegib, osimertinib, rolapitant, uridine triacetate, trabectedin, netupitant and palonosetron, belinostat, ibrutinib, olaparib, idelalisib, and ceritinib.

In certain aspects, the further therapeutic agent is an immune checkpoint inhibitor. Immune checkpoint inhibitors of interest include, but are not limited to, inhibitors (e.g., antibodies) that target PD-1, PD-L1, CTLA-4, TIM3, LAG3, or a member of the B7 family.

According to certain embodiments, the antibody that is administered as part of the antibody therapy, the inhibitory immune receptor inhibitor, and a further therapeutic agent are administered according to a dosing regimen approved for individual use. In some embodiments, the administration of the further therapeutic agent permits the antibody that is administered as part of the antibody therapy, the inhibitory immune receptor inhibitor, or both, administered to the individual to be administered according to a dosing regimen that involves one or more lower and/or less frequent doses, and/or a reduced number of cycles as compared with that utilized when the antibody that is administered as part of the antibody therapy, the inhibitory immune receptor inhibitor, or both, is administered without administration of the further therapeutic agent. In certain aspects, the administration of the antibody that is administered as part of the antibody therapy, the inhibitory immune receptor inhibitor, or both, permits the further therapeutic agent administered to the individual to be administered according to a dosing regimen that involves one or more lower and/or less frequent doses, and/or a reduced number of cycles as compared with that utilized when the further therapeutic agent is administered without administration of the antibody that is administered as part of the antibody therapy, the inhibitory immune receptor inhibitor, or both.

### Compositions

As summarized above, aspects of the present disclosure include pharmaceutical compositions. According to certain embodiments, a pharmaceutical composition of the present disclosure includes an inhibitory immune receptor inhibitor, a therapeutic antibody, and a pharmaceutically acceptable carrier. The pharmaceutical compositions generally include a therapeutically effective amount of the inhibitory immune receptor inhibitor and the therapeutic antibody.

Any of the inhibitory immune receptor inhibitors described above may be present in a pharmaceutical composition of the present disclosure. For example, the inhibitory immune receptor inhibitor may be any of the antibodies or small molecules described above. When the inhibitory immune receptor inhibitor is an antibody, the antibody may be an IgG (e.g., an IgG1, IgG2, IgG3 or IgG4 antibody), a single chain Fv (scFv), Fab, (Fab)2, (scFv′)2, or the like. The antibody may be a monoclonal antibody, a humanized antibody, a human antibody, etc.

An inhibitory immune receptor inhibitor present in a pharmaceutical composition of the present disclosure may specifically bind to an inhibitory immune receptor present on an immune cell selected from a natural killer (NK) cell, a macrophage, a monocyte, a neutrophil, a dendritic cell, a T cell, a B cell, a mast cell, a basophil, and an eosinophil. In certain aspects, the inhibitory immune receptor inhibitor specifically binds to a sialic acid-binding Ig-like lectin (Siglec) receptor (e.g., Siglec-7, Siglec-9, or both).

According to certain embodiments, the therapeutic antibody present in the pharmaceutical composition specifically binds to a tumor-associated antigen or a tumor-specific antigen. In certain aspects, the therapeutic antibody present in the pharmaceutical composition induces antibody-dependent cellular cytotoxicity (ADCC). In some embodiments, the therapeutic antibody present in the pharmaceutical composition specifically binds to an antigen selected from human epidermal growth factor receptor 2 (HER2), CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), and neuropilin-1 (NRP1). In certain aspects, the therapeutic antibody present in the pharmaceutical composition is selected from trastuzumab, cetuximab, daratumumab, girentuximab, panitumumab, ofatumumab, and rituximab.

The inhibitory immune receptor inhibitor and therapeutic antibody can be incorporated into a variety of formulations for therapeutic administration. More particularly, the inhibitory immune receptor inhibitor and therapeutic antibody can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the inhibitory immune receptor inhibitor and therapeutic antibody suitable for administration to the individual (e.g., suitable for human administration) are generally sterile and may further be free of detectable pyrogens or other contaminants contraindicated for administration to a patient according to a selected route of administration.

In pharmaceutical dosage forms, the inhibitory immune receptor inhibitor and therapeutic antibody can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the inhibitory immune receptor inhibitor and therapeutic antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The inhibitory immune receptor inhibitor and therapeutic antibody can be formulated for parenteral (e.g., intravenous, intra-arterial, intraosseous, intramuscular, intracerebral, intracerebroventricular, intrathecal, subcutaneous, etc.) administration. In certain aspects, the inhibitory immune receptor inhibitor and therapeutic antibody is formulated for injection by dissolving, suspending or emulsifying the conjugate in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the inhibitory immune receptor inhibitor and therapeutic antibody may be prepared by mixing the inhibitory immune receptor inhibitor and therapeutic antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the inhibitory immune receptor inhibitor and therapeutic antibody may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the inhibitory immune receptor inhibitor and therapeutic antibody against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the inhibitory immune receptor inhibitor and therapeutic antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

As summarized above, the present disclosure provides kits. According to certain embodiments, the kits include any of the compositions of the present disclosure. In certain aspects, the kits include a pharmaceutical composition including an inhibitory immune receptor inhibitor (e.g., any of the inhibitory immune receptors described elsewhere herein) and instructions for using the composition in combination with an antibody therapy being administered to an individual (e.g., an antibody therapy that includes administration of a therapeutic antibody that induces ADCC). The kits of the present disclosure find use, e.g., in practicing the methods of the present disclosure.

Kits for practicing the subject methods may include a quantity of the compositions, present in unit dosages, e.g., ampoules, or a multi-dosage format. As such, in certain embodiments, the kits may include one or more (e.g., two or more) unit dosages (e.g., ampoules) of a composition that includes an inhibitory immune receptor inhibitor, or an inhibitory immune receptor inhibitor and a therapeutic antibody (e.g., a therapeutic antibody that induces ADCC). In any of the compositions that include an inhibitory immune receptor inhibitor, the compositions may include one, two or more inhibitory immune receptor inhibitors, e.g., two inhibitory immune receptor inhibitors, such as a Siglec-7 inhibitor and a Siglec-9 inhibitor, a Siglec-7 inhibitor and a PD-1 inhibitor, a Siglec-9 inhibitor and a PD-1 inhibitor, or the like. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular inhibitory immune receptor inhibitor employed, the effect to be achieved, and the pharmacodynamics associated with the inhibitory immune receptor inhibitor, the therapeutic antibody, or both, in the subject. In yet other embodiments, the kits may include a single multi dosage amount of the composition.

Components of the kits may be present in separate containers, or multiple components may be present in a single container. For example, in a kit that includes both an inhibitory immune receptor inhibitor and a therapeutic antibody, the inhibitory immune receptor inhibitor and therapeutic antibody may be provided in the same composition (e.g., in one or more containers) or may be provided in separate compositions in separate containers. Suitable containers include individual tubes (e.g., vials), one or more wells of a plate (e.g., a 96-well plate, a 384-well plate, etc.), or the like.

According to certain embodiments, a kit of the present disclosure includes instructions for using the compositions to treat an individual in need thereof. For example, a kit may include instructions for using the inhibitory immune receptor inhibitor in combination with a therapeutic antibody that induces ADCC (which antibody may or may not be present in the kit) to potentiate ADCC in an individual in need thereof. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Potentiation of Natural Killer (NK) Cell-Mediated Antibody-Dependent Cellular Cytotoxicity (ADCC) by Treatment with Inhibitory Immune Receptor Blocking Antibodies

Natural Killer cells were purified from human peripheral blood mononuclear cells isolated from whole blood. They were then treated with the relevant receptor-blocking antibody (Siglec 7, Siglec 9 or NKG2D) at 5 microgram/mL for 1 hour at 37° C. They were then added to BT-20 or Ramos cells at ratios of 4:1 (NK:target cell) along with the therapeutic antibody (trastuzumab or rituximab) at 10 nM and allowed to incubate for 4 hours. After 4 hours, the cell mixtures were pelleted and the supernatant tested for levels of lactate dehydrogenase (LDH) released from lysed cells using a commercial LDH detection kit. From these values were subtracted measured levels of spontaneously released LDH from NK and target cells, and then these levels were compared to controls in which all of the target cells were lysed using a detergent to give a percent cytotoxicity.

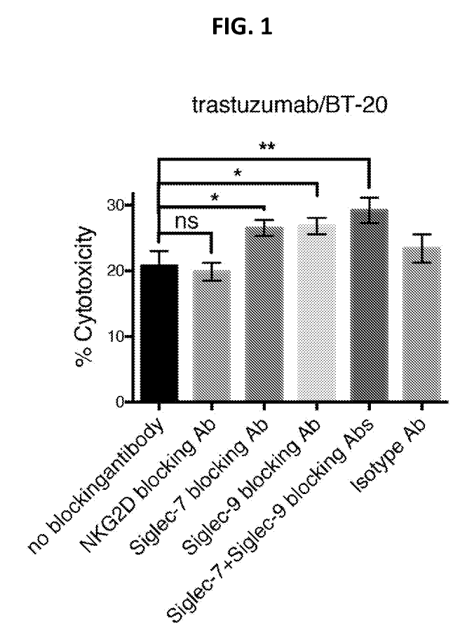

BT-20 (triple negative breast cancer) cells being treated with trastuzumab were subjected to the following conditions: (1) no blocking antibody; (2) Natural Killer Group 2D (NKG2D) blocking antibody (anti-NKG2D mAb (clone 149810) available from R&D Systems); (3) Siglec-7 blocking antibody (anti-Siglec-7 mAb (clone S7.7) available from Biolegend®); (4) Siglec-9 blocking antibody (anti-Siglec-9 mAb (clone K8) available from Biolegend®); (5) Siglec-7 blocking antibody and Siglec-9 blocking antibody; and (6) incubation with an isotype antibody. As shown in FIG. 1, the ADCC effect of trastuzamab was potentiated when the cells were co-treated with Siglec-7 blocking antibody, Siglec-9 blocking antibody, and a combination of Siglec-7 blocking antibody and Siglec-9 blocking antibody.

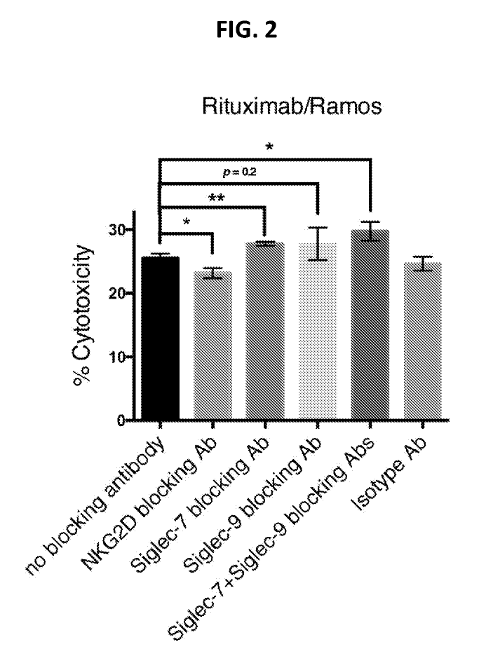

Ramos (B lymphocyte Burkitt's lymphoma) cells being treated with rituximab were subjected to the following conditions: (1) no blocking antibody; (2) Natural Killer Group 2D (NKG2D) blocking antibody (anti-NKG2D mAb (clone 149810) available from R&D Systems); (3) Siglec-7 blocking antibody (anti-Siglec-7 mAb (clone S7.7) available from Biolegend®); (4) Siglec-9 blocking antibody (anti-Siglec-9 mAb (clone K8) available from Biolegend®); (5) Siglec-7 blocking antibody and Siglec-9 blocking antibody; and (6) incubation with an isotype antibody. As shown in FIG. 2, the ADCC effect of rituximab was potentiated when the cells were co-treated with Siglec-7 blocking antibody, Siglec-9 blocking antibody, and a combination of Siglec-7 blocking antibody and Siglec-9 blocking antibody.

The data demonstrates a role for inhibitory immune receptors (in this example, Siglec receptors) in both trastuzumab- and rituximab-mediated ADCC.

### Example 2—Expression of Siglec Ligands

The expression levels of Siglec ligands on BT-20 and Ramos cells was determined by flow cytometry. For detection of Siglec ligands, cells were incubated with a soluble fusion protein that included the extracellular domain of Siglec-7 or Siglec-9 fused to a fragment crystallizable (Fc) antibody fragment (Sig-Fc), followed by incubation with a fluorescent labeled anti-Fc secondary antibody and detection by flow cytometry. More specifically, Siglec-Fc fusion proteins were pre-complexed at 5 μg/ml Sig-Fc and 4 μg/ml anti-Fc secondary antibody and incubated with cells for 30 min at 4° C. Cells were then washed 3 times and flow cytometry was performed. Separately, cells are treated with the anti-Fc secondary antibody (same 4 μg/ml), then washed as above and flow cytometry performed.

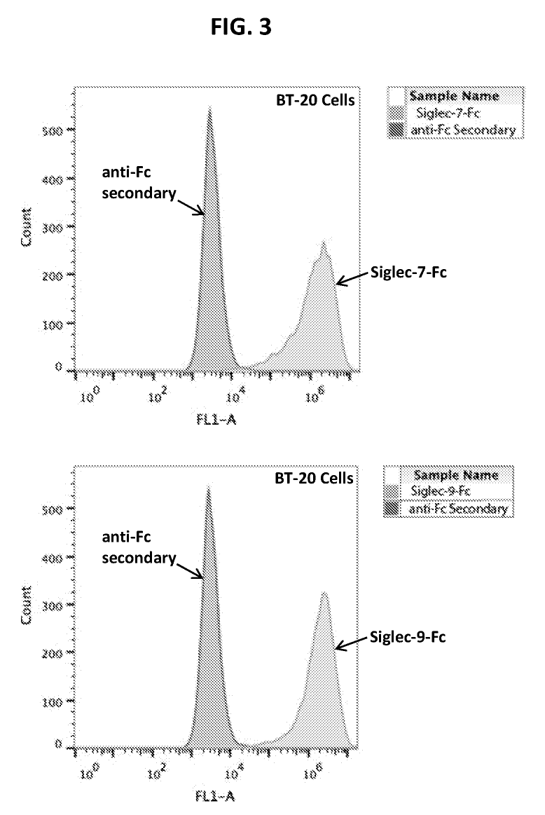

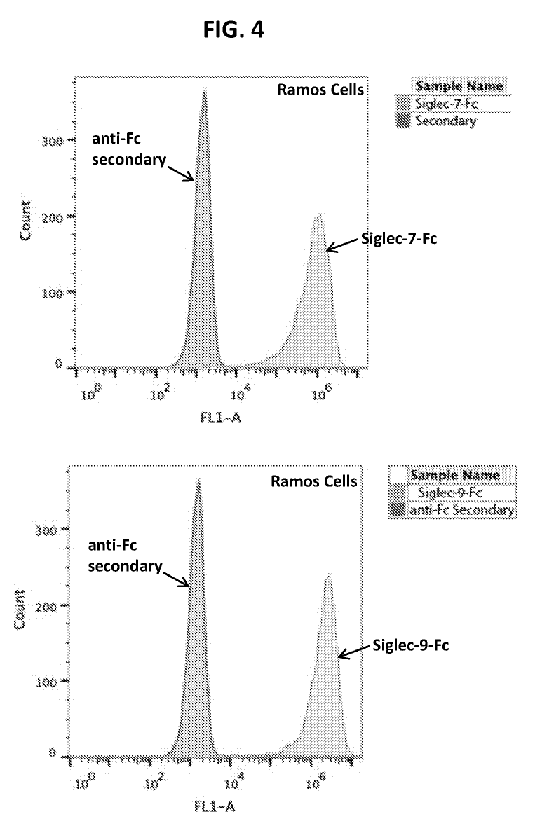

Expression levels of Siglec-7 ligands (top) and Siglec-9 ligands (bottom) on BT-20 cells is shown in FIG. 3. Expression levels of Siglec-7 ligands (top) and Siglec-9 ligands (bottom) on Ramos cells is shown in FIG. 4.

The observed increase in fluorescence of the Siglec-Fc fusion-treated cells over the secondary antibody-only treated cells indicates that binding was due to the Siglec-Fc fusion protein, and not the secondary reagent alone. That roughly three orders of magnitude more signal was observed in the Siglec-Fc fusion-treated cells indicates that both BT-20 cells and Ramos cells are rich in Siglec ligands, and provides a basis for why treatment with Siglec blocking antibodies is effective in potentiating ADCC as demonstrated in Example 1 above.

### Example 3—Siglec-7 Ligand Abundance Predicts Increase in ADCC by a Trastuzumab-Sialidase Conjugate

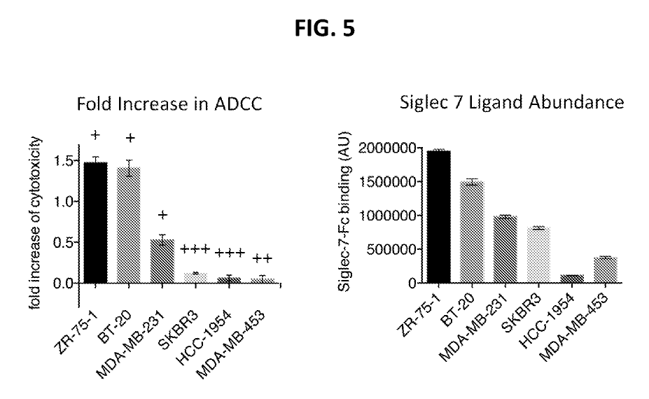

Purified NK cells (isolated as in Example 1), were mixed with the indicated target cell type at a ratio of 4:1, trastuzumab was added to a concentration of 10 nM and the cells allowed to react at 37° C. for 4 hours. In parallel, the same experiment was setup in which to this mixture of cells was added sialidase-trastuzumab conjugate to a concentration of 10 nM. At the end of 4 hours, the cell mixtures were pelleted and percent cytotoxicity was measured as in Example 1. A ‘fold increase in cytotoxicity’ was calculated by calculating the quotient of cytotoxicity of sialidase-pretreated target cells to the cytotoxicity of untreated cells minus 1. Using flow cytometry and the labeling technique described in Example 2, the abundance of both Her2 and cell-surface Siglec-7 ligands was calculated on these same target cell lines. Ranking cell lines from highest to lowest Siglec-7 ligand abundance nearly matches that of fold increase in ADCC cytotoxicity, whereas Her2 levels roughly negatively correlate with fold increase in cytotoxicity. HER2 expression level is indicated by number of ‘+’s. ADCC potentiation is most pronounced for HER2-low cell line, e.g., as seen in comparing MDA-MB-231 cells to SKBR3 cells (FIG. 5).

Notwithstanding the appended claims, the present disclosure is also defined by the following clauses:

1. A method, comprising:

administering to an individual receiving an antibody therapy an inhibitory immune receptor inhibitor.

2. The method according to Clause 1, wherein the inhibitory immune receptor inhibitor is an antibody or a small molecule.
3. The method according to Clause 2, wherein the inhibitory immune receptor inhibitor is an antibody.
4. The method according to Clause 3, wherein the antibody is selected from the group consisting of: an IgG, a single chain Fv (scFv), Fab, F(ab′)₂, or (scFv′)₂.
5. The method according to Clause 4, wherein the antibody is an IgG.
6. The method according to Clause 5, wherein the IgG is an IgG1, IgG2, IgG3, or IgG4.
7. The method according to any one of Clauses 3 to 6, wherein the antibody is a monoclonal antibody.
8. The method according to any one of Clauses 3 to 7, wherein the antibody is a humanized or human antibody.
9. The method according to any one of Clauses 1 to 8, wherein the inhibitory immune receptor inhibitor inhibits an inhibitory immune receptor present on an immune cell selected from the group consisting of: a natural killer (NK) cell, a macrophage, a monocyte, a neutrophil, a dendritic cell, a T cell, a B cell, a mast cell, a basophil, and an eosinophil.
10. The method according to any one of Clauses 1 to 9, wherein the inhibitory immune receptor is a sialic acid-binding Ig-like lectin (Siglec) receptor.
11. The method according to Clause 10, wherein the inhibitory immune receptor inhibitor inhibits Siglec-7.
12. The method according to Clause 10, wherein the inhibitory immune receptor inhibitor inhibits Siglec-9.
13. The method according to any one of Clauses 1 to 12, wherein the individual has cancer and the method is for treating the cancer.
14. The method according to Clause 13, wherein the individual has breast cancer, ovarian cancer, gastric cancer, colon cancer, renal carcinoma, or a combination thereof.
15. The method according to Clause 13 or Clause 14, wherein the individual is receiving an antibody therapy that comprises administering to the individual an antibody that specifically binds to a tumor-associated antigen.
16. The method according to Clause 13 or Clause 14, wherein the individual is receiving an antibody therapy that comprises administering to the individual an antibody that specifically binds to a tumor-specific antigen.
17. The method according to Clause 13 or Clause 14, wherein the individual is receiving an antibody therapy that comprises administering to the individual an antibody that specifically binds an antigen selected from the group consisting of: human epidermal growth factor receptor 2 (HER2), CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), and neuropilin-1 (NRP1).
18. The method according to any one of Clauses 13 to 17, wherein the individual is receiving an antibody therapy that comprises administering to the individual an antibody that induces antibody-dependent cellular cytotoxicity (ADCC).
19. The method according to any one of Clauses 1 to 11, wherein the individual is receiving an antibody therapy that comprises administering to the individual an antibody selected from the group consisting of: trastuzumab, cetuximab, daratumumab, girentuximab, panitumumab, ofatumumab, and rituximab.
20. The method according to any one of Clauses 1 to 19, comprising determining the abundance of one or more inhibitory immune receptor ligands present on cells targeted by the antibody therapy prior to administering the inhibitory immune receptor inhibitor.
21. The method according to Clause 20, wherein the inhibitory immune receptor inhibitor is administered to the individual only upon determining that the abundance of the one or more inhibitory immune receptor ligands on cells targeted by the antibody therapy exceeds a threshold abundance level.
22. The method according to Clause 20 or Clause 21, wherein the abundance of the one or more inhibitory immune receptor ligands is determined in vivo.
23. The method according to Clause 22, wherein the abundance of the one or more inhibitory immune receptor ligands is determined by in vivo imaging.
24. The method according to Clause 20 or Clause 21, wherein the abundance of the one or more inhibitory immune receptor ligands is determined in vitro.
25. The method according to Clause 24, wherein the abundance of the one or more inhibitory immune receptor ligands is determined on a biopsy sample.
26. The method according to Clause 25, wherein determining the abundance of one or more inhibitory immune receptor ligands comprises incubating cells of the biopsy sample with a reagent comprising an extracellular domain of the inhibitory immune receptor.
27. The method according to Clause 26, wherein the reagent comprises a fusion protein comprising the extracellular domain of the inhibitory immune receptor.
28. The method according to Clause 27, wherein the fusion protein comprises a fragment crystallizable (Fc) antibody fragment.
29. The method according to any one of Clauses 20 to 28, wherein the one or more inhibitory immune receptor ligands comprises a Siglec-7 ligand.
30. The method according to any one of Clauses 20 to 29, wherein the one or more inhibitory immune receptor ligands comprises a Siglec-9 ligand.
31. The method according to any one of Clauses 1 to 30, wherein the inhibitory immune receptor inhibitor is administered to the individual prior to the onset of the antibody therapy.
32. The method according to any one of Clauses 1 to 31, wherein the inhibitory immune receptor inhibitor is administered to the individual concurrently with the antibody therapy.
33. The method according to Clause 32, wherein the inhibitory immune receptor inhibitor and the antibody of the antibody therapy are administered to the individual present in a same pharmaceutical composition.
34. A pharmaceutical composition, comprising:

an inhibitory immune receptor inhibitor;

a therapeutic antibody; and

a pharmaceutically acceptable carrier.

35. The pharmaceutical composition of Clause 34, wherein the inhibitory immune receptor inhibitor is a small molecule.
36. The pharmaceutical composition of Clause 34, wherein the inhibitory immune receptor inhibitor is an antibody.
37. The pharmaceutical composition of Clause 36, wherein the antibody is selected from the group consisting of: an IgG, a single chain Fv (scFv), Fab, F(ab′)₂, or (scFv)₂.
38. The pharmaceutical composition of Clause 36 or Clause 37, wherein the antibody is a monoclonal antibody.
39. The pharmaceutical composition of any one of Clauses 36 to 38, wherein the antibody is a humanized or human antibody.
40. The pharmaceutical composition of any one of Clauses 34 to 39, wherein the inhibitory immune receptor inhibitor specifically binds to an inhibitory immune receptor present on an immune cell selected from the group consisting of: a natural killer (NK) cell, a macrophage, a monocyte, a neutrophil, a dendritic cell, a T cell, a B cell, a mast cell, a basophil, and an eosinophil.
41. The pharmaceutical composition of any one of Clauses 34 to 40, wherein the inhibitory immune receptor inhibitor specifically binds to a sialic acid-binding Ig-like lectin (Siglec) receptor.
42. The pharmaceutical composition of Clause 41, wherein the inhibitory immune receptor inhibitor specifically binds to Siglec-7.
43. The pharmaceutical composition of Clause 41, wherein the inhibitory immune receptor inhibitor specifically binds to Siglec-9.
44. The pharmaceutical composition of any one of Clauses 34 to 43, comprising two or more inhibitory immune receptor inhibitors.
45. The pharmaceutical composition of Clause 44, comprising two inhibitory immune receptor inhibitors selected from the group consisting of: a Siglec-7 inhibitor, a Siglec-9 inhibitor, and a PD-1 inhibitor.
46. The pharmaceutical composition of any one of Clauses 34 to 45, wherein the therapeutic antibody specifically binds to a tumor-associated antigen.
47. The pharmaceutical composition of any one of Clauses 34 to 45, wherein the therapeutic antibody specifically binds to a tumor-specific antigen.
48. The pharmaceutical composition of any one of Clauses 34 to 47, wherein the therapeutic antibody induces antibody-dependent cellular cytotoxicity (ADCC).
49. The pharmaceutical composition of any one of Clauses 34 to 45, wherein the therapeutic antibody specifically binds to an antigen selected from the group consisting of: human epidermal growth factor receptor 2 (HER2), CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), and neuropilin-1 (NRP1).
50. The pharmaceutical composition of any one of Clauses 34 to 45, wherein the therapeutic antibody is selected from the group consisting of: trastuzumab, cetuximab, daratumumab, girentuximab, panitumumab, ofatumumab, and rituximab.
51. A kit comprising the pharmaceutical composition of any one of Clauses 34 to 50.
52. The kit of Clause 51, wherein the kit comprises the pharmaceutical composition in one or more unit dosages.
53. The kit of Clause 51 or Clause 52, comprising instructions for using the composition to treat an individual in need thereof by antibody-dependent cellular cytotoxicity (ADCC).
54. A kit, comprising:
a pharmaceutical composition comprising an inhibitory immune receptor inhibitor; and
instructions for using the composition in combination with an antibody therapy being administered to an individual.
   55. The kit of Clause 54, wherein the kit comprises the pharmaceutical composition in one or more unit dosages.
   56. The kit of Clause 54 or 55, wherein the inhibitory immune receptor inhibitor is an antibody.
   57. The kit of any one of Clauses 54 to 56, wherein the inhibitory immune receptor inhibitor inhibits Siglec-7.
   58. The kit of any one of Clauses 54 to 56, wherein the inhibitory immune receptor inhibitor inhibits Siglec-9.
   59. The kit of any one of Clauses 54 to 58, wherein the pharmaceutical composition comprising an inhibitory immune receptor inhibitor comprises two or more inhibitory immune receptor inhibitors.
   60. The kit of Clause 59, wherein the pharmaceutical composition comprising an inhibitory immune receptor inhibitor comprises two or more inhibitory immune receptor inhibitors selected from the group consisting of: a Siglec-7 inhibitor, a Siglec-9 inhibitor, and a PD-1 inhibitor.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method, comprising:
administering to an individual receiving an antibody therapy an inhibitory immune receptor inhibitor.

2. The method according to claim 1, wherein the inhibitory immune receptor inhibitor is an antibody or a small molecule.

3. The method according to claim 2, wherein the inhibitory immune receptor inhibitor is an antibody.

4. The method according to claim 3, wherein the antibody is selected from the group consisting of: an IgG, a single chain Fv (scFv), Fab, F(ab′)₂, or (scFv′)₂.

5. The method according to claim 4, wherein the antibody is an IgG.

6. The method according to claim 5, wherein the IgG is an IgG1, IgG2, IgG3, or IgG4.

7. The method according to any one of claims 3 to 6, wherein the antibody is a monoclonal antibody.

8. The method according to any one of claims 3 to 7, wherein the antibody is a humanized or human antibody.

9. The method according to any one of claims 1 to 8, wherein the inhibitory immune receptor inhibitor inhibits an inhibitory immune receptor present on an immune cell selected from the group consisting of: a natural killer (NK) cell, a macrophage, a monocyte, a neutrophil, a dendritic cell, a T cell, a B cell, a mast cell, a basophil, and an eosinophil.

10. The method according to any one of claims 1 to 9, wherein the inhibitory immune receptor is a sialic acid-binding Ig-like lectin (Siglec) receptor.

11. The method according to claim 10, wherein the inhibitory immune receptor inhibitor inhibits Siglec-7.

12. The method according to claim 10, wherein the inhibitory immune receptor inhibitor inhibits Siglec-9.

13. The method according to any one of claims 1 to 12, wherein the individual has cancer and the method is for treating the cancer.

14. The method according to claim 13, wherein the individual has breast cancer, ovarian cancer, gastric cancer, colon cancer, renal carcinoma, or a combination thereof.

15. The method according to claim 13 or claim 14, wherein the individual is receiving an antibody therapy that comprises administering to the individual an antibody that specifically binds to a tumor-associated antigen.

16. The method according to claim 13 or claim 14, wherein the individual is receiving an antibody therapy that comprises administering to the individual an antibody that specifically binds to a tumor-specific antigen.

17. The method according to claim 13 or claim 14, wherein the individual is receiving an antibody therapy that comprises administering to the individual an antibody that specifically binds an antigen selected from the group consisting of: human epidermal growth factor receptor 2 (HER2), CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), and neuropilin-1 (NRP1).

18. The method according to any one of claims 13 to 17, wherein the individual is receiving an antibody therapy that comprises administering to the individual an antibody that induces antibody-dependent cellular cytotoxicity (ADCC).

19. The method according to any one of claims 1 to 11, wherein the individual is receiving an antibody therapy that comprises administering to the individual an antibody selected from the group consisting of: trastuzumab, cetuximab, daratumumab, girentuximab, panitumumab, ofatumumab, and rituximab.

20. The method according to any one of claims 1 to 19, comprising determining the abundance of one or more inhibitory immune receptor ligands present on cells targeted by the antibody therapy prior to administering the inhibitory immune receptor inhibitor.

21. The method according to claim 20, wherein the inhibitory immune receptor inhibitor is administered to the individual only upon determining that the abundance of the one or more inhibitory immune receptor ligands on cells targeted by the antibody therapy exceeds a threshold abundance level.

22. The method according to claim 20 or claim 21, wherein the abundance of the one or more inhibitory immune receptor ligands is determined in vivo.

23. The method according to claim 22, wherein the abundance of the one or more inhibitory immune receptor ligands is determined by in vivo imaging.

24. The method according to claim 20 or claim 21, wherein the abundance of the one or more inhibitory immune receptor ligands is determined in vitro.

25. The method according to claim 24, wherein the abundance of the one or more inhibitory immune receptor ligands is determined on a biopsy sample.

26. The method according to claim 25, wherein determining the abundance of one or more inhibitory immune receptor ligands comprises incubating cells of the biopsy sample with a reagent comprising an extracellular domain of the inhibitory immune receptor.

27. The method according to claim 26, wherein the reagent comprises a fusion protein comprising the extracellular domain of the inhibitory immune receptor.

28. The method according to claim 27, wherein the fusion protein comprises a fragment crystallizable (Fc) antibody fragment.

29. The method according to any one of claims 20 to 28, wherein the one or more inhibitory immune receptor ligands comprises a Siglec-7 ligand.

30. The method according to any one of claims 20 to 29, wherein the one or more inhibitory immune receptor ligands comprises a Siglec-9 ligand.

31. The method according to any one of claims 1 to 30, wherein the inhibitory immune receptor inhibitor is administered to the individual prior to the onset of the antibody therapy.

32. The method according to any one of claims 1 to 31, wherein the inhibitory immune receptor inhibitor is administered to the individual concurrently with the antibody therapy.

33. The method according to claim 32, wherein the inhibitory immune receptor inhibitor and the antibody of the antibody therapy are administered to the individual present in a same pharmaceutical composition.

34. A pharmaceutical composition, comprising:
an inhibitory immune receptor inhibitor;
a therapeutic antibody; and
a pharmaceutically acceptable carrier.

35. The pharmaceutical composition of claim 34, wherein the inhibitory immune receptor inhibitor is a small molecule.

36. The pharmaceutical composition of claim 34, wherein the inhibitory immune receptor inhibitor is an antibody.

37. The pharmaceutical composition of claim 36, wherein the antibody is selected from the group consisting of: an IgG, a single chain Fv (scFv), Fab, F(ab′)₂, or (scFv′)₂.

38. The pharmaceutical composition of claim 36 or claim 37, wherein the antibody is a monoclonal antibody.

39. The pharmaceutical composition of any one of claims 36 to 38, wherein the antibody is a humanized or human antibody.

40. The pharmaceutical composition of any one of claims 34 to 39, wherein the inhibitory immune receptor inhibitor specifically binds to an inhibitory immune receptor present on an immune cell selected from the group consisting of: a natural killer (NK) cell, a macrophage, a monocyte, a neutrophil, a dendritic cell, a T cell, a B cell, a mast cell, a basophil, and an eosinophil.

41. The pharmaceutical composition of any one of claims 34 to 40, wherein the inhibitory immune receptor inhibitor specifically binds to a sialic acid-binding Ig-like lectin (Siglec) receptor.

42. The pharmaceutical composition of claim 41, wherein the inhibitory immune receptor inhibitor specifically binds to Siglec-7.

43. The pharmaceutical composition of claim 41, wherein the inhibitory immune receptor inhibitor specifically binds to Siglec-9.

44. The pharmaceutical composition of any one of claims 34 to 43, comprising two or more inhibitory immune receptor inhibitors.

45. The pharmaceutical composition of claim 44, comprising two inhibitory immune receptor inhibitors selected from the group consisting of: a Siglec-7 inhibitor, a Siglec-9 inhibitor, and a PD-1 inhibitor.

46. The pharmaceutical composition of any one of claims 34 to 45, wherein the therapeutic antibody specifically binds to a tumor-associated antigen.

47. The pharmaceutical composition of any one of claims 34 to 45, wherein the therapeutic antibody specifically binds to a tumor-specific antigen.

48. The pharmaceutical composition of any one of claims 34 to 47, wherein the therapeutic antibody induces antibody-dependent cellular cytotoxicity (ADCC).

49. The pharmaceutical composition of any one of claims 34 to 45, wherein the therapeutic antibody specifically binds to an antigen selected from the group consisting of: human epidermal growth factor receptor 2 (HER2), CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, an integrin, C—X—C chemokine receptor type 4 (CXCR4), cytotoxic T-lymphocyte-associated protein 4 (CTLA-4), and neuropilin-1 (NRP1).

50. The pharmaceutical composition of any one of claims 34 to 45, wherein the therapeutic antibody is selected from the group consisting of: trastuzumab, cetuximab, daratumumab, girentuximab, panitumumab, ofatumumab, and rituximab.

51. A kit comprising the pharmaceutical composition of any one of claims 34 to 50.

52. The kit of claim 51, wherein the kit comprises the pharmaceutical composition in one or more unit dosages.

53. The kit of claim 51 or claim 52, comprising instructions for using the composition to treat an individual in need thereof by antibody-dependent cellular cytotoxicity (ADCC).

54. A kit, comprising:
a pharmaceutical composition comprising an inhibitory immune receptor inhibitor; and
instructions for using the composition in combination with an antibody therapy being administered to an individual.

55. The kit of claim 54, wherein the kit comprises the pharmaceutical composition in one or more unit dosages.

56. The kit of claim 54 or 55, wherein the inhibitory immune receptor inhibitor is an antibody.

57. The kit of any one of claims 54 to 56, wherein the inhibitory immune receptor inhibitor inhibits Siglec-7.

58. The kit of any one of claims 54 to 56, wherein the inhibitory immune receptor inhibitor inhibits Siglec-9.

59. The kit of any one of claims 54 to 58, wherein the pharmaceutical composition comprising an inhibitory immune receptor inhibitor comprises two or more inhibitory immune receptor inhibitors.

60. The kit of claim 59, wherein the pharmaceutical composition comprising an inhibitory immune receptor inhibitor comprises two or more inhibitory immune receptor inhibitors selected from the group consisting of: a Siglec-7 inhibitor, a Siglec-9 inhibitor, and a PD-1 inhibitor.
